(19) United States
(12) Patent Application Publication
(10) Pub. No.: US 2013/0171252 A1
(43) Pub. Date: Jul. 4, 2013

(54) Formulation Comprising a Type B Lantibiotic

(71) Applicants: Antony Nicholas Appleyard, Hertfordshire (GB); Sjoerd Nicolaas Wadman, Hertfordshire (GB)

(72) Inventors: Antony Nicholas Appleyard, Hertfordshire (GB); Sjoerd Nicolaas Wadman, Hertfordshire (GB)

(73) Assignee: NOVACTA BIOSYSTEMS LIMITED, Hertfordshire (GB)

(21) Appl. No.: 13/810,162

(22) PCT Filed: Jul. 12, 2011
(86) PCT No.: PCT/GB2011/001046

§ 371 Date: Jan. 14, 2013

Related U.S. Application Data
(60) Provisional application No. 61/364,088, filed on Jul. 14, 2010.

(51) Int. Cl. A61K 38/12

(52) U.S. Cl. CPC A61K 38/12
USPC 424/451; 514/2.9; 206/462

(57) ABSTRACT

Described is a pharmaceutical formulation of a capsule for oral delivery of a type B lantibiotic to the stomach comprising a hard gelatine, HPMC or starch capsule, and a type B lantibiotic of formula (I): wherein X is —NH(CH₂)_{q}NH₂ and q is an integer 2 to 12.

This application is related to U.S. 61/364,088 filed 14 Jul. 2010; the contents of which are incorporated herein by reference in their entirety.

The present disclosure relates to a formulation for oral delivery of a type B lantibiotic of formula (I), in particular a rapidly disintegrating capsule which delivers the lantibiotic to the stomach and use of the same in therapy, in particular in the treatment of Clostridium difficile infection. The disclosure also extends to methods of preparing said formulations.

Type B lantibiotics (globular peptides) are known, for example from WO 2007/083112. Formulations of lantibiotics, such as nisin (a lanthocin), are known from U.S. Pat. No. 5,985,823 and U.S. Pat. No. 5,304,540. These cases describe a formulation that maintains its integrity through the gastrointestinal tract and then permits release of a lanthocin into the colon. They include appropriately coated tablets or granules or capsules for oral administration, wherein said coating affords maintenance of the integrity of the dosage form during passage through the stomach and small intestine and permits release of the active ingredient in the desired region of the gastrointestinal tract (lower small intestine to upper large intestine).

The disclosure herein provides a pharmaceutical formulation of a capsule for oral delivery of a type B lantibiotic to the stomach comprising:
a gelatine, HPMC or starch capsule;
a type B lantibiotic of formula (I):

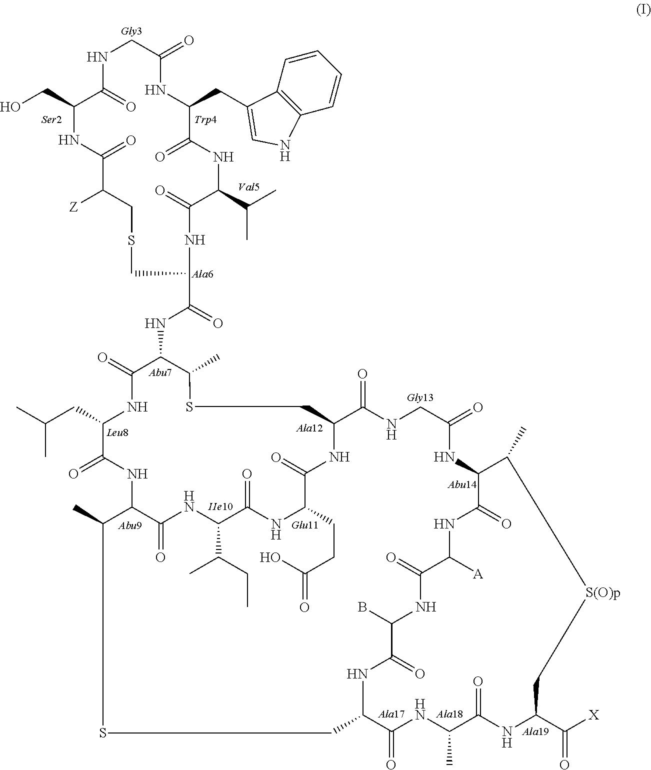

wherein:
A together with the carbon to which it is attached and the alpha-nitrogen and alpha-carbonyl represents a proteinogenic amino acid residue selected from leucine, isoleucine and valine;
B together with the carbon to which it is attached and the alpha-nitrogen and alpha-carbonyl represents a proteinogenic amino acid residue selected from leucine, isoleucine and valine;
X is —NH(CH₂)_{q}NH₂;
q is an integer 2 to 12;

### Z is —NR¹R²;

R¹ is H or C₁₋₄ alkyl,
R² is H, an amino acid or C₁₋₄ alkyl, and
p is 0 or 1, or
a pharmaceutically acceptable salt or solvate thereof,
wherein the capsule releases the type B lantibiotic into the stomach within, for example 30 minutes, 25 minutes, 20 minutes, 15 minutes, such as 10 minutes of oral delivery.

### BRIEF DESCRIPTION OF THE FIGURES

FIG. 1 shows a comparison in stability for compound 1 (top line) and nisin (bottom line) in SIF over time. It can be seen that compound 1 (labelled compound of formula (II)) is essentially stable in SIF;

FIG. 2 shows photographs of a capsule of the invention (comprising compound 1) dissolving over time. The vials are test samples at 2 min and 3 min (FIG. 2a); 5-6 min, 10 min, and 15 min (FIG. 2b). A test capsule (without compound 1) in SGF is shown after 15 min (FIG. 2c);

FIG. 3 shows the dissolution of compound 1 (compound of formula (II)) over time. Compound 1 in SGF appears to have reached saturation dissolution after 3 minutes;

FIG. 4 shows the stability of compound 1 (referred to as NVB 302) in SIF as peak area expressed as a percentage with respect to time zero peak area, plotted against sampling time.

Type B lantibiotics are not degraded substantially by conditions found in the stomach and the intestines and do not require to be delivered in an enteric coated formulation to ensure that the active ingredient is delivered safely to the colon. However, surprisingly the inventors have found that the compounds of formula (I) are more soluble in stomach acid than in gastrointestinal fluid. The present inventors believe it is advantageous to deliver the lantibiotic to the stomach, such that it can dissolve and/or disperse readily and flow through to the intestines in a diluted (dissolved and/or dispersed form).

In contrast, releasing the lantibiotic in the intestines or colon, which is a drier environment, may in fact result in inferior distribution of the same. In addition the intestinal fluid has a higher pH than gastric fluid. The lower pH of the stomach may assist the dispersion of the lantibiotic.

The present disclosure provides a formulation that allows the lantibiotic, in particular substantially all of the dose in the capsule, to be released into the stomach and certainly be release by the time of passing into the duodenum.

Whilst not wishing to be bound by theory, it is hypothesised that the high stability of type B lantibiotics to the conditions of the stomach and/or intestines is due in part to the globular structure. Nevertheless, folding of the peptide and/or the formation of small agglomerations (or globules) may contribute to this stability.

Substantially all in the context of the present specification, means an amount approximately equivalent to the intended dose in the capsule, for example at least 60, 65, 70, 75, 0, 85, 90, 91, 92, 93, 94, 95, 96, 97, 98, 99 or 100% w/w of the lantibiotic contained in the capsule.

In one embodiment the lantibiotic is released in 9 or less minutes, for example 8, 7, 6, 5 or less minutes after administration.

The capsules employed in the formulation herein should not be coated to delay the release of the lantibiotic contained therein.

In one embodiment the thickness of the capsule shell is about 0.1 mm.

Gelatine dissolves in the conditions provided in the stomach. However, gelatine is one of the proteins derived from animals and is not suitable for use with all patient populations. The consistency of the capsule shell may be modified by the inclusion of excipients such as glycerol and/or sorbitol.

In one embodiment the gelatine capsule is hard gelatine.

In one embodiment the gelatine capsule is soft gelatine.

In one embodiment the capsule employed is a Swedish orange hard capsule.

HPMC capsule as employed herein is intended to refer to hydroxypropyl methyl cellulose capsule, for example as prepared by routine methods or as described in US 2010/0168410.

Alternatively, the capsules may be starch for example capsules prepared from corn starch.

In one embodiment the capsule size is selected from 000, 00E, 00, 0E, 1, 2, 3 or 4, such as 00.

The content of the capsule may be a solid, a liquid or a paste.

In one embodiment the capsule is a hard capsule and, for example contains a solid content.

In one embodiment a preservative may be employed in the formulation.

In one embodiment each capsule of the formulation contains between 10 mg and 500 mg of lantibiotic, such as 50 mg to 350 mg.

In one embodiment at least two capsules are employed to administer a “single” dose in the range 100 mg to 1000 mg, such as 150 mg to 500 mg or 50 mg to 300 mg, in particular 60, 70, 80, 90, 100, 110, 120, 130, 140, 150, 160, 170, 180, 190, 200, 210, 220, 230, 240, 250, 260, 270, 280, 290 or 300 mg. A single dose as used in the latter context is intended to refer to a dose given on one occasion, for example when the capsules are administered concomitantly or sequential one immediately after the other.

The lantibiotic may be provided as a salt for example an addition salt formed from inorganic or organic acids which form non-toxic salts including lactobionate, mandelate (including (S)-(+)-mandelate, (R)-(−)-mandelate and (R,S)-mandelate), hydrochloride, hydrobromide, hydroiodide, sulfate, bisulfate, nitrate, phosphate, hydrogen phosphate, glutamate, acetate, trifluoroacetate, maleate, malate, fumarate, lactate, tartrate, citrate, formate, gluconate, succinate, ethyl succinate (4-ethoxy-4-oxo-butanoate), pyruvate, oxalate, oxaloacetate, saccharate, benzoate, glucolate, glucamate (including N-methyl glucamate and N-ethyl glucamate) glucurinate, alkyl or aryl sulphonates (eg methanesulphonate, ethanesulphonate, benzenesulphonate or p-toluenesulphonate), and isethionate.

Other example of pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine, N-ethyl-D-glucamine and N-methyl-D-glucamine.

Salts may be employed to optimize the solubility of the compounds of the present disclosure.

In one embodiment the lantibiotic compound is provided with a free amine at the C-terminal (i.e. as the free base). The compounds employed in the formulation of the present invention are amphoteric and may be present as zwitter ions.

In one embodiment the lantibiotic is in the form of a solvate, such as a hydrate.

In one embodiment the lantibiotic material employed in the formulation of the present invention is amorphous.

In one embodiment the lantibiotic material employed in the formulation has been subjected to a pre-treatment step of lyophilisation, for example in the preparation of a salt.

In one embodiment the lantibiotic material employed has been spray-dried, for example to provide a material with suitable flow properties. In one embodiment the lantibiotic is spray dried with one or more excipients to provide particles that are agglomerations or simple mixtures (admixtures) of the lantibiotic and the excipients.

In one embodiment the formulation filled into the capsule consists or consists essentially of the lantibiotic of formula (I) or a salt or solvate thereof.

In one embodiment the formulation filled into the capsule comprised the lantibiotic of formula (I) and a pharmaceutically acceptable excipient.

Pharmaceutically acceptable excipients include microcrystalline cellulose, lactose, mannitol, starch, such as pre-gelatinised starch, talc, lubricants such as magnesium stearate, stearic acid, glycerol and polyethylene glycol, buffering agents such as sodium carbonate and the like.

In one embodiment the formulation filled into the capsule comprises one or more excipients independently selected from microcrystalline cellulose, lactose, sodium citrate, calcium carbonate, calcium sulphate, dibasic calcium phosphate and glycine, mannitol, pregelatinised starch, corn starch, potato starch, disintegrants such as sodium starch glycollate, croscarmellose sodium and certain complex silicates, and granulation binders such as polyvinylpyrrolidone.

Alkyl in the context of the present disclosure refers to straight chain or branched chain alkyl, for example methyl, ethyl, propyl, isopropyl, n-butyl or t-butyl.

In one embodiment p is 1. In one embodiment p is 0.

In one aspect A together with the carbon to which it is attached and the alpha-nitrogen and alpha-carbonyl is leucine and B together with the carbon to which it is attached and the alpha-nitrogen and alpha-carbonyl is valine.

In one embodiment A together with the carbon to which it is attached and the alpha-nitrogen and alpha-carbonyl is valine and B together with the carbon to which it is attached and the alpha-nitrogen and alpha-carbonyl is isoleucine.

In one embodiment A together with the carbon to which it is attached and the alpha-nitrogen and alpha-carbonyl is valine and B together with the carbon to which it is attached and the alpha-nitrogen and alpha-carbonyl is valine.

In one embodiment A together with the carbon to which it is attached and the alpha-nitrogen and alpha-carbonyl is leucine and B together with the carbon to which it is attached and the alpha-nitrogen and alpha-carbonyl is isoleucine.

In one embodiment R¹ is H.

In one embodiment R² is H.

In one embodiment R² is the L or D isomer form of an amino acid residue. In one embodiment R² is the L or D isomer form of —C(O)CH(CH₃)NH₂.

In one embodiment R² is an amino acid residue selected from alanine, cysteine, aspartic acid, glutamic acid, phenylalanine, glycine, histidine, isoleucine, lysine, leucine, methionine, asparagine, proline, glutamine, arginine, serine, threonine, valine, tryptophan and tyrosine.

In one embodiment R² is an amino acid residue selected from phenylalanine, tyrosine and alanine (i.e. —C(O)CH(CH₃)NH₂).

In one embodiment Z is —NH₂.

In one aspect A is —CH₂CH(CH₃)₂ and B is —CH(CH₃)₂ and Z is —NH₂.

In one embodiment q is 2, 3, 4, 5, 6, 7, 8, 9, 10, 11 or 12, such as 2, 3, 7, 9 or 12, in particular 7, 9 or 12. In one embodiment q is 7. In another embodiment q is 9 or 12.

In one embodiment q is 3 to 12 or 3 to 8.

Each and every compatible combination of the embodiments described above is explicitly disclosed herein, as if each and every combination was individually and explicitly recited.

In one aspect the disclosure provides a compound of formula (II):

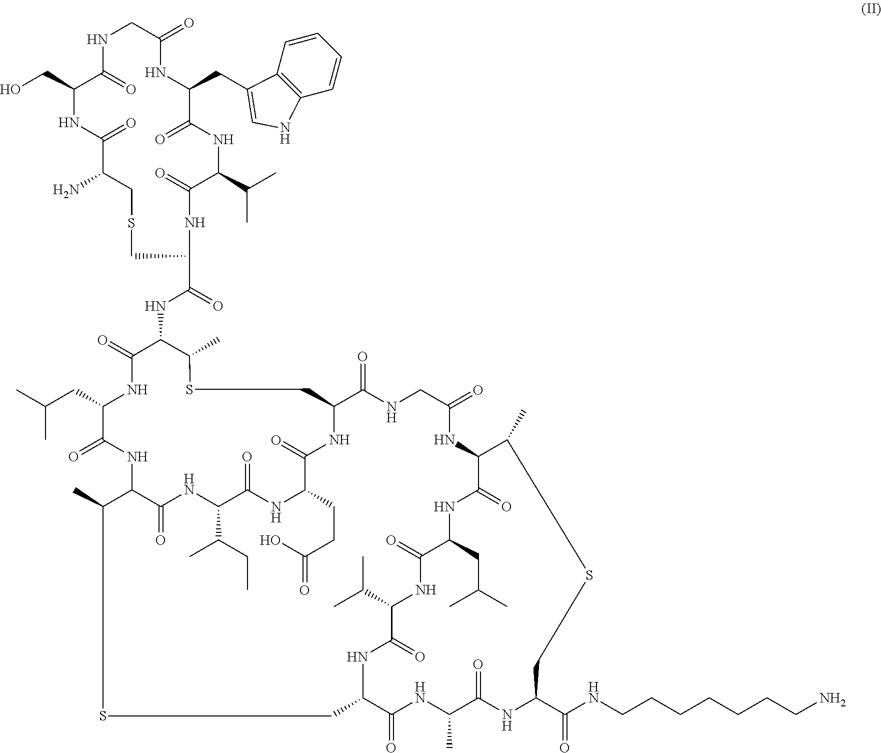 [SMILES: CCC(C)[C@@H]1NC(=O)C2NC(=O)[C@H](CC(C)C)NC(=O)[C@H](NC(=O)[C@H]3CSC[C@H](N)C(=O)N[C@@H](CO)C(=O)NCC(=O)N[C@@H](Cc4c[nH]c5ccccc45)C(=O)N[C@@H](C(C)C)C(=O)N3)[C@H](C)SC[C@H](NC(=O)[C@H](CCC(=O)O)NC1=O)C(=O)NCC(=O)N[C@@H]1C(=O)N[C@@H](CC(C)C)C(=O)N[C@@H](C(C)C)C(=O)N[C@@H](CS[C@H]2C)C(=O)N[C@@H](C)C(=O)N[C@H](C(=O)NCCCCCCCN)CS[C@H]1C]

or a pharmaceutically acceptable salt, hydrate or solvate thereof.

In one embodiment the compound of formula (I) or (II) comprises 5-10% w/w of water.

In one embodiment the formulation according to the present invention comprises a compound of formula (I) or (II) and an antioxidant, for example butylated hydroxytoluene. Suitable amounts of antioxidant, such as butylated hydroxyl toluene include 10% w/w or less, for example 9, 8, 7, 6, 5, 4, 3, 2 or 1% w/w of the final formulation.

In one embodiment the formulation of the present disclosure has a moisture content of less than 8%, such as less than 7, 6, 5, 4, 3, 2 or 1% w/w after capsule filing.

In one embodiment capsules of the invention are filled under controlled humidity conditions. Thus there is provided a method of preparing a solid dose form according to the invention comprising the step of filling a compound of formula (I) or (II) or a composition comprising the same into a capsule under controlled humidity conditions.

In one embodiment the formulation according to the disclosure has a shelf life of about 2 years, when stored under appropriate conditions. In particular the formulation is physically stable after storage (e.g. the flow properties of the contents of the capsules are unchanged and/or there is no aggregation in the formulation and/or the disintegration time of the capsule remains substantially unchanged and/or water ingress is minimised) and the lantibiotic therein is chemically stable over said period.

In one embodiment at the end of the shelf life, after storage under appropriate conditions for example as defined on the label, the moisture content of the formulation is less than 12% w/w or less such as 10% w/w or less.

In one embodiment the capsules of the present disclosure are packed into blister foil packs, for example foil/foil packs or foil laminate packs. Suitable package is known to those working in the relevant field.

The compounds employed in the formulations of the present disclosure are advantageous because they have very high antibacterial activity against one or more strains of C. difficile, for example when activity is measured by a standard test such as minimum inhibitory concentrations (MICs), generally the compounds of the disclosure have an MIC of 16 μg/mL or less such as 4 μg/mL or less, in particular 2 μg/mL or lower against one or more C. difficile strains. Furthermore, certain compounds herein have very high activity against a number of common strains of C. difficile.

Additionally, the compounds of formula (I) and (II) are particularly suited to administration to humans and animals because they have low antibacterial activity against the naturally occurring healthy intestinal flora found in the body. In the case of treatment of diarrhoea induced by a microbial infection such as C. difficile it is expected that a reduced recurrence of symptoms will be observed after treatment with the present compounds in comparison to treatment with known antibiotics because of the ability of the natural flora to survive the treatment with the present compounds. In particular the compounds herein show very low activity against Bacteroides fragilis, Bacteroides thetaiotaomicron, Lactobacillus rhamnosus, and moderately low activity against Peptostreptococcus anaerobius and Bifidobacterium adolescentis.

What is more, when delivered orally the compounds of the disclosure are not absorbed systemically, which allows a relatively high concentration of the active ingredient to be delivered to the target in the colon/intestines. Thus because there is no systemic delivery of the compounds when administered orally, this may minimise any potential side effects for patients.

C. difficile infection and/or overgrowth is a common problem for patients during hospitalisation. It presents a genuine burden to the health care system and may be life threatening to vulnerable patients such as elderly patients.

Thus in one aspect there is provided use of a formulation according to the present disclosure in treatment, particularly in the treatment of humans and/or animals, such as treatment of microbial infection, more specifically C. difficile infection.

The formulations of the disclosure are particularly suitable for administration (for example in the treatment or prophylaxis of C. difficile infection) to patients on proton pump inhibitors or with hypochlorhydria. These patients are more suceptable to C. difficile infection and reinfection via the faecal-oral route because the bacteria may survive passage through the more favourable conditions in the stomach of these patients and subsequently colonise the colon. Release of the compounds of formula (I) and (II) in stomach is likely to eliminate bacteria in the stomach thereby preventing infection or re-infection of the colon.

Thus in one embodiment there is provided a method of treating a patient population with a formulation according the present invention, wherein the patient population is characterized by taking proton pump inhibitors or having hypochlorhydria.

In one aspect there is provided a formulation as described herein comprising a compound of formula (I) or (II) for the manufacture of a medicament for the treatment of microbial infections such as C. difficile infection, in particular diarrhoea or colitis associated therewith.

In one aspect there is provided a method of treatment comprising the step of administering a therapeutically effective amount of a compound of formula (I), such as compound of formula (II), or a pharmaceutical composition containing the same as described herein to a patient (human or animal) in need thereof, for example for the treatment of an infection/illness or disease as described herein.

In the context of this specification “comprising” is to be interpreted as “including”. Aspects of the invention comprising certain elements are also intended to extend to alternative embodiments “consisting” or “consisting essentially” of the relevant elements.

Embodiments of the invention may be combined as technically appropriate.

### EXAMPLES

### Compound 1

### Deoxyactagardine B (1,7-diaminoheptane) monocarboxamide

Deoxyactagardine B (2.5 g), 1,7-diaminoheptane (0.52 g) and diisopropylethylamine (0.44 mL) were dissolved in dry dimethylformamide (10 mL). A solution of benzotriazole-1-yl-oxy-tris-pyrrolidino-phosphonium hexafluorophosphate (PyBOP) (1.04 g) in dry dimethylformamide (5 mL) was added portionwise over 2 h. The reaction was followed by analytical HPLC (See Table 1) and PyBOP was added until the starting material had been consumed.

TABLE 1
Analytical HPLC conditions for the separation of lantibiotic
(e.g. actagardine, actagardine B, or deoxy-actagardine B)
and diaminoalkane derivatised products.

| Column: | Zorbax 5μ C18(2) 150 × 4.6 mm | | | | |
|---|---|---|---|---|---|
| Mobile Phase A: | 30% Acetonitrile in 20 mM potassium phosphate | | | | |
| | buffer pH 7.0 | | | | |
| Mobile Phase B: | 65% Acetonitrile in 20 mM potassium phosphate | | | | |
| | buffer pH 7.0 | | | | |
| Flow rate: | 1 ml/min | | | | |
| Gradient: | Time 0 min | 100% | A | 0% | B |
| | Time 10 min | 0% | A | 100% | B |
| | Time 11 min | 0% | A | 100% | B |
| | Time 11.2 min | 100% | A | 0% | B |

| | |
|---|---|
| Cycle time 15 min | |
| Injection volume: | 10 μl |
| Detection: | 210 nm |

The crude reaction mixture was poured into 30% aqueous methanol and the resulting solution was loaded on to a Varian Bond Elut C18 column (30 g). The column was then washed sequentially with 50%, 60%, 70%, 80%, 90% aqueous methanol, with most of the desired material eluting in the 70% fraction. Column chromatography on silica gel (eluent dichloromethane:ethanol:ammonia 10:8:1) gave material of >90% purity by U.V. at 210 nm. Yield 1.4 g. Mass calc (M+2H)⁺² 993. found 992.91.

The product was analysed by ¹³C NMR spectroscopy at 500 MHz (solvent D₃ acetonitrile:water in a ratio 7:3). A peak listing is provided in Table 2.

TABLE 2
Carbon 13 peak listing for Compound 1.

| PEAK | [ppm] |
|---|---|
| 1 | 181.3149 |
| 2 | 175.3919 |
| 3 | 174.8404 |
| 4 | 174.6462 |
| 5 | 174.3911 |
| 6 | 174.2256 |
| 7 | 174.0976 |
| 8 | 173.8498 |
| 9 | 173.4321 |
| 10 | 173.3003 |
| 11 | 173.1919 |
| 12 | 172.8374 |
| 13 | 172.5363 |
| 14 | 172.5226 |
| 15 | 171.6244 |
| 16 | 171.403 |
| 17 | 171.2443 |
| 18 | 171.2186 |
| 19 | 137.4317 |
| 20 | 128.2591 |
| 21 | 125.4133 |
| 22 | 122.6186 |
| 23 | 120.101 |
| 24 | 119.489 |
| 25 | 119.2236 |
| 26 | 112.6147 |
| 27 | 110.3448 |
| 28 | 62.6628 |
| 29 | 62.3103 |
| 30 | 61.9417 |
| 31 | 60.0459 |
| 32 | 59.2589 |
| 33 | 57.6883 |
| 34 | 57.5602 |
| 35 | 57.1782 |
| 36 | 56.3394 |
| 37 | 55.779 |
| 38 | 55.1894 |
| 39 | 54.8993 |
| 40 | 54.8157 |
| 41 | 54.4243 |
| 42 | 53.0651 |
| 43 | 52.6472 |
| 44 | 51.5046 |
| 45 | 47.0088 |
| 46 | 44.8668 |
| 47 | 44.6775 |
| 48 | 44.5744 |
| 49 | 43.8023 |
| 50 | 42.6752 |
| 51 | 41.1394 |
| 52 | 40.7135 |
| 53 | 40.0986 |
| 54 | 36.7443 |
| 55 | 36.5221 |
| 56 | 36.0111 |
| 57 | 35.0293 |
| 58 | 33.5143 |
| 59 | 31.0095 |
| 60 | 30.9257 |
| 61 | 30.2204 |
| 62 | 29.4444 |
| 63 | 28.9958 |
| 64 | 28.1579 |
| 65 | 27.8264 |
| 66 | 27.3108 |
| 67 | 26.8943 |
| 68 | 26.6716 |
| 69 | 26.0067 |
| 70 | 25.6053 |
| 71 | 25.5072 |
| 72 | 23.0708 |
| 73 | 22.7664 |
| 74 | 22.7369 |
| 75 | 21.9216 |
| 76 | 20.7945 |
| 77 | 20.7139 |
| 78 | 20.5133 |
| 79 | 19.7487 |
| 80 | 19.6807 |
| 81 | 19.3537 |
| 82 | 18.6924 |
| 83 | 17.3511 |
| 84 | 16.1335 |
| 85 | 12.0709 |
| 86 | 1.8865 |
| 87 | 1.7212 |
| 88 | 1.5557 |
| 89 | 1.3899 |
| 90 | 1.2242 |
| 91 | 1.0588 |
| 92 | 0.8934 |

### Compound 2

### Preparation of the Methanesulfonate Salt of the Compound of Compound 1

For the purpose of obtaining solutions suitable for oral or intravenous dosing, the methanesulfonate salt of the compound of compound 1 was found to be suitable.

The compound of compound 1 was suspended in water and an excess of methanesulfonic acid was added to give a clear solution. Excess methanesulfonic acid was removed by loading the solution onto a Bond Elut C18 column that had been conditioned according to the manufacturer's instructions, washing the column thoroughly with water and eluting the methanesulfonate salt with methanol. The solvent was removed by evaporation leaving the methanesulfonate salt as a white powder.

The methanesulfonate salt of the compound of compound 1 was soluble at approximately 20 mg/mL in water.

### Compound 3

### (Alternative Route for Preparation of Compound of Compound 1): Deoxyactagardine B [7-(t-butoxycarbonylamido)-1-aminoheptane]monocarboxamide]

Was prepared employing the process described for compound 1 from Deoxyactagardine B and 7-(t-butoxycarbonylamido)-1-aminoheptane. 75% (M+2H)⁺² 1043. found 1044.11.

Compound 3 was treated with 4N aqueous hydrochloric acid for 3 h at room temperature, whereupon the mixture was neutralised to pH7 and purification was carried out as described for Example 1 to provide Compound 1. Yield: 65%.

### Compound 4

### Deoxyactagardine B (1,2-ethylene diamine) monocarboxamide

Was prepared from deoxyactagardine B and 1,2-ethylenediamine employing the method described for compound 1. Yield: 96%. Mass calc (M+2H)^{+2 958}. found 959.02.

### Compound 5

### Deoxyactagardine B (1,3-diaminopropane) monocarboxamide

Was prepared from deoxyactagardine B and 1,3-diaminopropane employing the method described for compound 1. Yield: 87%. Mass calc (M+2H)^{+2 965}. found 965.04.

### Compound 6

### Deoxyactagardine B (1,5-diaminopentane) monocarboxamide

Was prepared from deoxyactagardine B and 1,5-diaminopentane employing the method described for compound 1. Yield: 83%. Mass calc (M+2H)^{+2 979}. found 980.06.

### Compound 7

### Deoxyactagardine B (1,9-diaminononane) monocarboxamide

Was prepared from deoxyactagardine B and 1,9-diaminononane employing the method described for compound 1. Yield: 84%. Mass calc (M+2H)⁺² 1007. found 1007.51.

### Compound 8

### Deoxyactagardine B (1,12-diaminododecane) monocarboxamide)

Was prepared from deoxyactagardine B and 1,12-diaminododecane employing the method described for compound 1. Yield: 74%. Mass calc (M+2H)⁺² 1028. found 1027.51.

### Compound 9

### Actagardine (1,7-diaminoheptane) monocarboxamide

Was prepared from the amide coupling of Actagardine with 1,7-diaminoheptane employing the method described for compound 1. Yield: 59%. Mass calc (M+2H)⁺² 1001.0. found 1001.02.

### Compound 10

### Actagardine (1,3-diaminopropane) monocarboxamide

Was prepared by coupling actagardine with 1,3-diaminopropane utilising the procedure described for compound 1. Yield: 47%. Mass calc (M+H+ Na)⁺² 973.0. found 973.2.

### Compound 11

### Actagardine (1,4-diaminobutane) monocarboxamide

Was prepared by coupling actagardine with 1,4-diaminobutane utilising the procedure described for compound 1. Yield: 50%. Mass calc (M+H+ Na)⁺² 990.5. found 989.46.

### Antibacterial Activity of Type-B Lantibiotics

The compounds employed in the invention show antimicrobial activity in vitro and in vivo. They are active against Clostridium difficile and may have improved activity compared to deoxyactagardine B.

Susceptibility testing for Clostridium difficile strains was performed by two-fold serial antibiotic dilutions in Wilkins-Chalgren Anaerobe agar under anaerobic conditions. Vancomycin was included as a comparator drug. C. difficile cultures were inoculated onto pre-reduced Braziers (C.C.E.Y.) agar plates and grown at 37° C. for 48 hours under anaerobic conditions. Two to three colonies of the 48 hours cultures were inoculated into 5 ml of pre-reduced Schaedlers Broth and grown at 37° C. for 24 hours under anaerobic conditions. This culture was diluted with pre-reduced 0.9% NaCl to achieve the turbidity of the 0.5 McFarland standard and applied to the drug containing plates at a final inoculum of 105 cfu/spot. Drug-free growth control plates were included. The plates were incubated in the anaerobic chamber at 37° C. for 48 hours and examined for growth. The MIC was the lowest concentration of drug that completely inhibited growth or caused markedly reduction of growth as compared to growth on the drug-free plates.

TABLE 3
MIC data (μg/ml) for deoxyactagardine B (DAB), and derivatives
thereof. (The lower the value of the result the greater the activity of
the test compound.)

| | Compound Number | | | | | | |
|---|---|---|---|---|---|---|---|
| C. diff | | Comp 3 | Comp 5 | Comp 6 | Comp 1 | Comp 7 | Comp 8 |
| strain | DAB | (p = 2) | (p = 3) | (p = 5) | (p = 7) | (p = 9) | (p = 12) |
| 37779 | 4 | 2, 2 | 1, 1 | 2, 2 | 1, 0.5 | 2, 1 | 1, 2 |
| | | 2, 2 | 1, 1 | | 1, 1 | | 1, 1 |
| | | 2, 1 | 1, 1 | | 1, 1 | | |
| | | | 2, 2 | | 1, 1 | | |
| 19126 | 4 | 2, 1 | 2, 2 | 1, 1 | 1, 1 | 2, 1 | 1, 1 |
| | | 1, 2 | 1, 1 | | 0.5, 1 | | 2, 2 |
| | | 1, 1 | 1, 1 | | 1, 1 | | |
| | | | 2, 2 | | 1, 0.5 | | |
| B32 | 2 | 2, 2 | 2, 2 | 2, 2 | 1, 1 | 2, 2 | 2, 1 |
| E16 | 2 | 4, 2 | 1, 2 | 2, 2 | 1, 2 | 2, 2 | 2, 2 |
| P24 | 2 | 2, 2 | 2, 2 | 2, 2 | 1, 1 | 2, 1 | 2, 1 |
| 027SM | 2 | 2, 2 | 2, 2 | 2, 2 | 1, 1 | 2, 2 | 2, 2 |
| P62 | 2 | 2, 2 | 2, 2 | 2, 2 | 2, 1 | 2, 2 | 2, 2 |
| E101 | 2 | 2, 2 | 2, 2 | 2, 2 | 1, 1 | 2, 2 | 2, 2 |
| 027Can | 4 | 2, 2 | 1, 1 | 2, 1 | 0.5, 0.5 | 0.5 | 1, 1 |
| | | 1, 2 | 0.5, 0.5 | | 0.5, 0.5 | 1, 1 | 1, 1 |
| | | 0.5 | 1, 0.5 | | 1, 1 | | |
| | | 1 | 2, 2 | | 1, 0.5 | | |
| E4 | 2 | | | | | | |
| P49 | 2 | | | | | | |
| P59 | 2 | | | | | | |
| 630 | 4 | 1, 1 | 1, 1 | 1, 1 | 0.5, 0.5 | | 0.5, 0.5 |
| | | 1, 1 | 2, 2 | 1, 0.5 | 0.5, 1 | | 1, 1 |
| | | 1, 1 | 1, 1 | 1, 1 | 1, 1 | | |
| | | | | 1, 1 | 1, 1 | | |

### Stability of Type-B Lantibiotics in Intestinal Fluid

The lantibiotic-based compounds provided herein may have increased stability to enzymatic degradation compared to type-A lantibiotics, such as nisin. Particularly, the compounds may have improved stability to intestinal juices compared to type-A lantibiotics.

Nisin and the compound of compound 1 were tested for their susceptibility towards enzymatic digestion in the intestine using a simulated intestinal fluid (SIF). The SIF was based on the standard USP solutions for simulated intestinal fluids and its activity was confirmed against Bovine Serum Albumin (Hilger et al, Clin. Exp. Immunol. 2001, 123, 387-94). The compounds were incubated in SIF at 37° C. and their concentrations quantified by analytical HPLC (UV detection at 210 nm using the conditions outlined in Table 1).

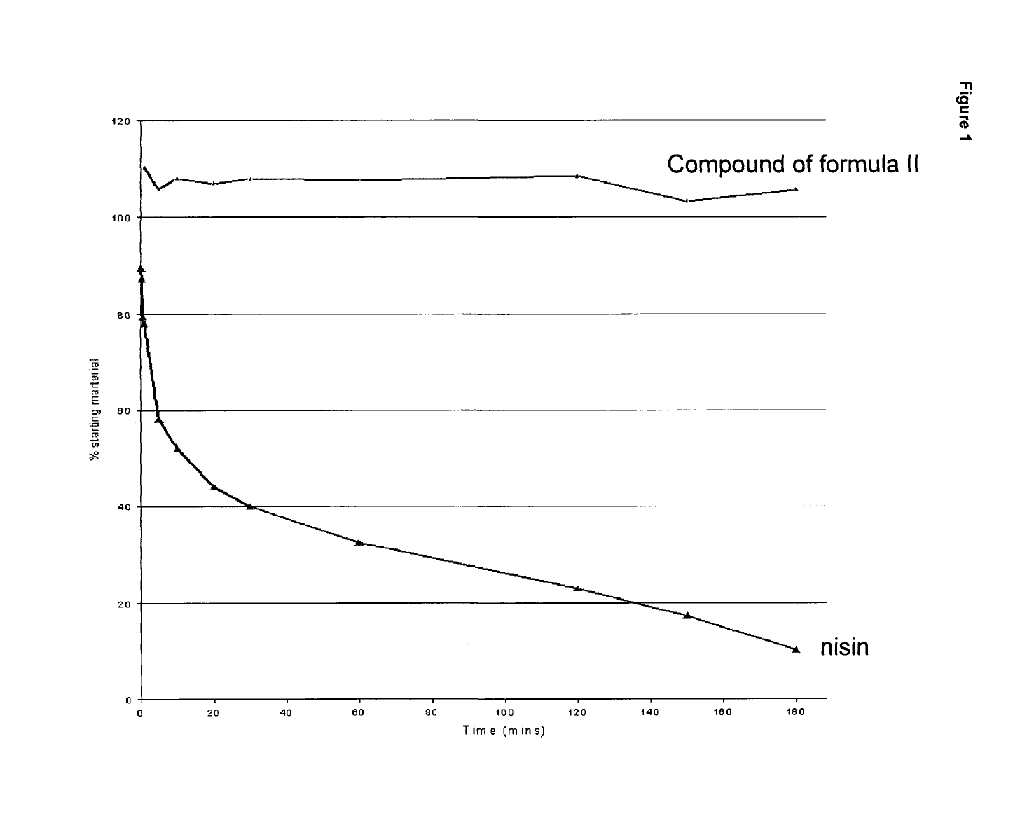

FIG. 1 shows that nisin was rapidly degraded in SIF with a half-life of approximately 15 to 20 minutes. The rapid degradation of nisin in this medium supports the observation that the clinical utility of nisin for the treatment of colonic infections is very limited unless the compound can be protected from degradative enzymes by means of careful formulation.

FIG. 1 also shows that the compound 1 (labelled compound of formula II) is essentially stable in SIF and likely to have suitable stability for treating colonic C. difficile infections.

What is more type B lantibiotics such as a compound of formula I is stable in SGF for up to 20 hours.

### Example 1

A hard gelatine capsule (size 00) was opened into two segments and compound 1 (50 mg) was weighed into the larger segment. The capsule segment containing compound 1 was sealed by inserting and closing the smaller segment over the larger capsule section. A hard gelatine capsule (size 00) containing up to 500 mg can be prepared employing this method.

### Dissolution Testing

The capsule of Example 1 was dropped into a 10 mL of simulated gastric fluid (SGF) at 37° C. The resulting mixture was stirred gently with a magnetic stir bar such that the capsule rotated slowly in the solution, such that it did not touch the stir bar. Samples of the solution/suspension (100 μL) were withdrawn at 1, 2, 3, 4, 5, 6, 7, 8, 9, 10, 15, 20, 25, 30 and 70 minutes after the addition of the capsule to SGF.

The samples were clarified using a bench top centrifuge to remove solid from the suspension. The clarified supernatants were diluted with 50% ethanol and then were analysed by HPLC.

The results show that compound 1 was completely released from the capsule into SGF within 4 minutes.

### Example 2

The solubility and dissolution behaviour of compound 1 contained in a hard gelatin capsule when suspended in simulated gastric fluid (SGF) has been monitored at 37° C.

Compound 1 (50 mg) was transferred into a size 00 hard gelatin capsule. This capsule represents the lowest dose that is likely to be administered in clinical trials. The capsule was dropped into 10 mL of SGF at 37° C. and the sample was intermittently swirled by hand. Dissolution was monitored visually and by HPLC over a one hour period. For the dissolution experiment monitored by HPLC, the sample was stirred continuously with a magnetic stirrer at 37° C. 100 ul samples were withdrawn from the dissolution experiment and were diluted with 900 ul water. The diluted sample was then centrifuged to remove solid material (mostly gelatin).

### Composition of SGF

| | |
|---|---|
| NaCl | 2 g |
| Pepsin | 3.2 g |
| HCl | 7 mL |
| Made to 1 L water | |

HPLC method used

### Column: Phenomenex Hyperclone 5μ C18 150×4.6 mm

### Mobile Phase A: 10% Acetonitrile/90% Water/0.15% TFA

### Mobile Phase B: 90% Acetonitrile/10% Water/0.15% TFA

Flow rate: 1 mL/min

### Gradient:

| | | | | |
|---|---|---|---|---|
| Time 0 min | 100% | A | 0% | B |
| Time 10 min | 0% | A | 100% | B |
| Time 11 min | 0% | A | 100% | B |
| Time 11.2 min | 100% | A | 0% | B |
| Cycle time 15 min | | | | |

Injection volume: 10 μL

### Detection: 210 nm

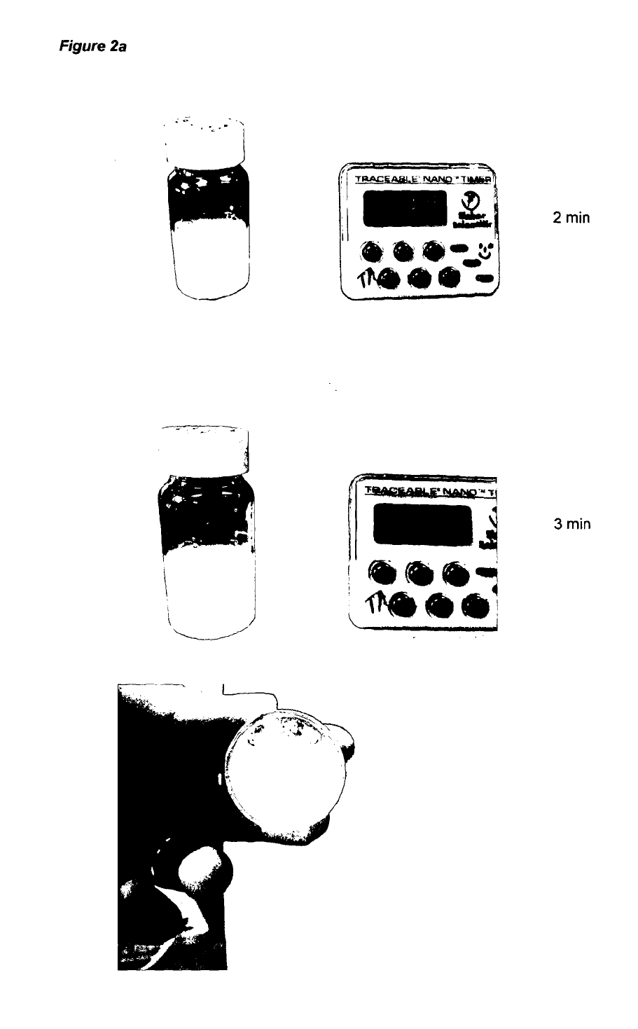

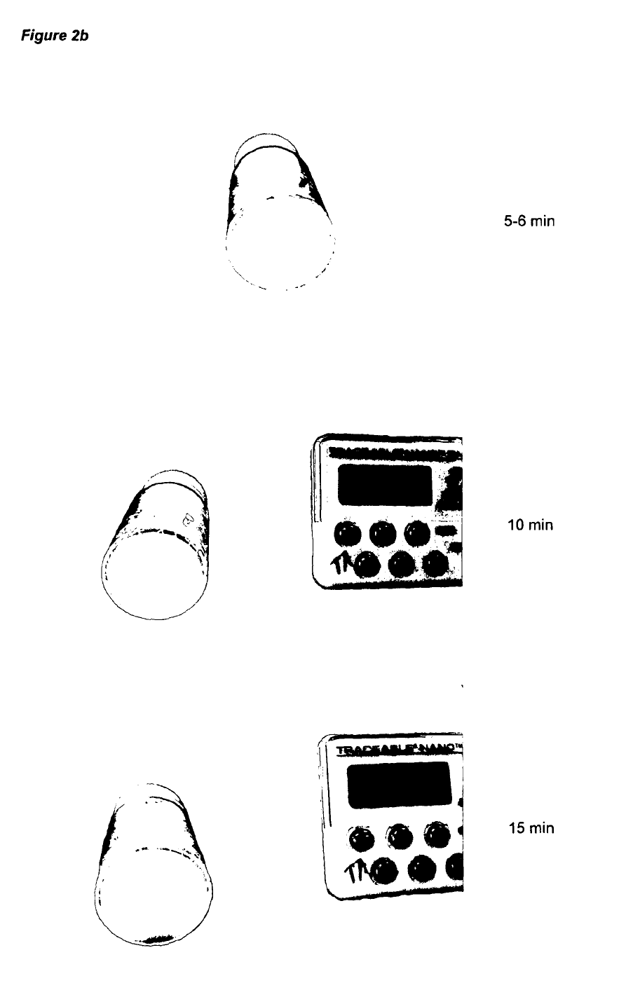

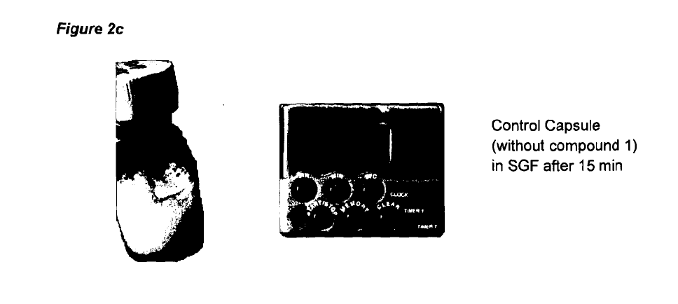

The capsule remains intact for approximately 1 minute. After 1 minute a hole forms in the capsule and drug begins to disperse. After 4 minutes the capsule forms a capsule/drug lump on the side of the glass vessel. After 10 minutes a small deposit of capsule remains and solid compound was not visible. The white suspension is formed mostly by the gelatin capsule (see control capsule below, FIG. 2)

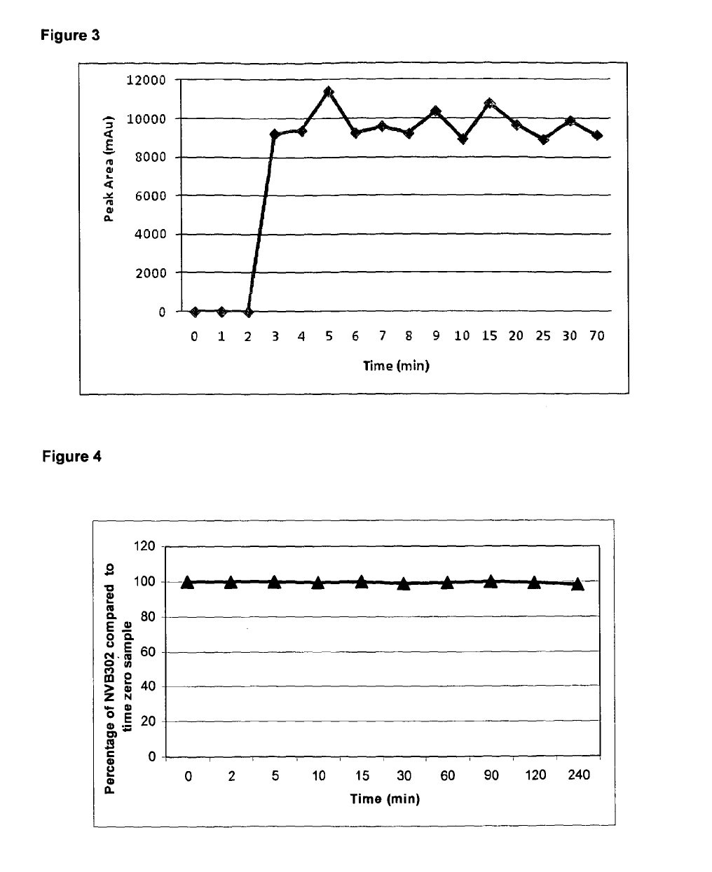

Compound 1 appears to have reached saturation dissolution after 3 minutes (FIG. 3). The variance in peak area response is partly due to poor chromatography due to the interaction with gelatin on the column. However, the results do indicate that the dissolution of compound 1 is rapid (within 3 minutes) and that the bulk of compound 1 dissolves in less than 5 minutes. The study demonstrates that compound 1 dissolves and disperses into SGF from a hard gelatin capsule.

Compound 1 and compound of formula (II) are used interchangeably herein.

### Example 3

10 mg of compound 1 was suspended in 250 μg/mL Simulated Intestinal Fluid (SIF), and was left on a shaker for 10 minutes, after which time suspension was observed. This was in contrast to an identical sample suspended in SGF which dissolved fully after the same period of time. This sample prepared at a target concentration of 40 mg/ml was centrifuged to remove sediment. The supernatant was then diluted to a target of 1 mg/ml with water and compound 1 content was compared against a 1 mg/mL standard prepared in 50% ethanol (aq) using a HPLC method of Example 2.

### Composition of SIF

Add the following items to a beaker in the order listed below

| | |
|---|---|
| KH₂PO₄ | 6.8 g |
| Water | 250 mL |
| 0.2N NaOH | 77 mL |
| Water | 500 mL |
| Pancreatin | 10 g |
| HCl/NaOH | to adjust to pH 6.8 |
| Make to 1 L water | (Final pH = 6.8) |

Compound 1 is soluble in SIF at lower than 0.8 mg/mL. This was in contrast to the solubility of compound 1 in SGF which was soluble at 40 mg/mL.

### Example 4

Compound 1 was incubated in SIF at 37° C. for up to 240 minutes. Aliquots were withdrawn from the test sample and diluted with water prior to analysis by HPLC (as described in Example 2). The compound 1 peak area was recorded and was expressed as a percentage with respect to the time zero sample. Over 240 minutes no significant reduction in compound 1 peak area was observed indicating that compound 1 is stable in SIF for 240 minutes (FIG. 4).

1.-38. (canceled)

39. A pharmaceutical formulation of a capsule for oral delivery of a type B lantibiotic to the stomach comprising:
a rapidly disintegrating capsule;
a type B lantibiotic of formula (I): 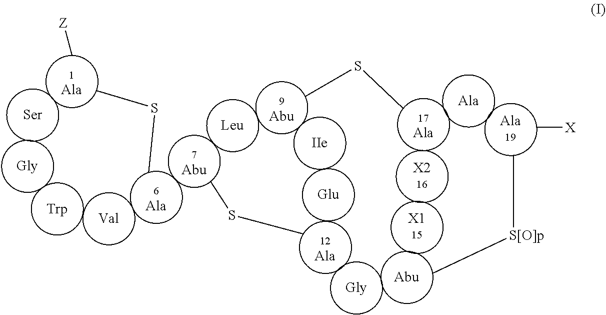
wherein
X1-X2 is selected from the group consisting of Leu-Leu, Leu-Ile, Leu-Val, Ile-Leu, Ile-Ile, Ile-Val, Val-Ile and Val-Leu;
X is —NH(CH₂)_{q}NH₂;
q is an integer 2 to 12;
Z is —NR¹R²;
R¹ is H or C₁₋₄ alkyl,
R² is H, an amino acid or C₁₋₄ alkyl, and
p is 0 or 1, or
a pharmaceutically acceptable salt or solvate thereof,
wherein the capsule releases the type B lantibiotic into the stomach.

40. A pharmaceutical formulation according to claim 39, wherein said formulation allows at least 60% of the type B lantibiotic contained in the capsule to be released into the stomach and substantially all of the type B lantibiotic to be released by the time of passing into the duodenum.

41. A pharmaceutical formulation according to claim 39, wherein the capsule releases the type B lantibiotic into the stomach within 15 minutes.

42. A pharmaceutical formulation according to claim 39, wherein the thickness of the capsule shell is about 0.1 mm.

43. A pharmaceutical formulation according to claim 39, wherein the formulation is not coated.

44. A pharmaceutical formulation according to claim 39, wherein the capsule is a gelatine, HPMC or starch capsule.

45. A pharmaceutical formulation according to claim 39 wherein X1-X2 is Leu-Val.

46. A pharmaceutical formulation according to claim 39, wherein X1-X2 is Val-Ile.

47. A pharmaceutical formulation according claim 39, wherein R² is the L or D isomer form of an amino acid residue.

48. A pharmaceutical formulation according to claim 39, wherein R² is an amino acid residue selected from the group consisting of Phe, Tyr and Ala.

49. A pharmaceutical formulation according to claim 48, wherein R² is Ala.

50. A pharmaceutical formulation according to claim 39, wherein q is any one of 2, 3, 4, 5, 6, 7, 8, 9, 10, 11 and 12.

51. A pharmaceutical formulation according to claim 39, wherein q is any one of 2, 3, 7, 9 and 12.

52. A pharmaceutical formulation according to claim 39, wherein q is any one of 7, 9 and 12.

53. A pharmaceutical formulation according claim 39, wherein Z is NH₂.

54. A pharmaceutical formulation according to claim 39, wherein p is 1.

55. A pharmaceutical formulation according to claim 39, wherein the compound of formula (II) is deoxyactagardine B (1,7-diaminoheptane) monocarboxamide,
or a pharmaceutically acceptable salt or solvate thereof.

56. A pharmaceutical formulation according to claim 39, wherein the lantibiotic is released in any one of 9, 8, 7, 6, 5 or less minutes after oral administration.

57. A pharmaceutical formulation according to claim 39, wherein the lantibiotic employed in the formulation is amorphous.

58. A pharmaceutical formulation according to claim 39, wherein the lantibiotic employed in the formulation has been subjected to a pre-treatment step of lyophilisation.

59. A pharmaceutical formulation according to claim 39, wherein the lantibiotic employed has been spray-dried.

60. A pharmaceutical formulation according to claim 39, and one or more pharmaceutically acceptable excipients.

61. A pharmaceutical formulation according to claim 39, wherein the lantibiotic is spray dried with one or more excipients to provide particles that are agglomerations or simple mixtures of the lantibiotic and the excipients.

62. A pharmaceutical formulation according to claim 39, wherein said formulation has a moisture content of any one of 0, 1, 2, 3, 4, 5, 6, 7, 8, 9, 10, 11 and 12% w/w after capsule filling.

63. A pharmaceutical formulation according to claim 39, wherein said formulation has a shelf life of about 2 years, when stored under appropriate conditions.

64. A pharmaceutical formulation according to claim 63, wherein said formulation is physically stable and the lantibiotic therein is chemically stable over said period.

65. A pharmaceutical formulation according to claim 39, wherein at the end of the shelf life, the moisture content of the formulation is less than 12% w/w after storage under appropriate conditions.

66. A pharmaceutical formulation according to claim 39, wherein the capsules are packed into blister foil/foil or foil/laminate packs or high density polyethylene container, in particular fitted with a hygroscopic sachet.

67. A method of treating a microbial infection, the method comprising administering a therapeutically effective amount of a compound of formula (I) according to claim 39 to a patient in need thereof.

68. A method according to claim 67, wherein the microbial infection is a Clostridium difficile infection.

69. A method according to claim 68, wherein the Clostridium difficile infection is in the colon and/or lower intestines.

70. A method according to claim 67, wherein the microbial infection is small intestine bacterial overgrowth.

71. A method according to claim 70 for treating ulcerative colitis.

72. A method according to claim 71 for treating irritable bowel syndrome.

73. A method according to claim 72 for preventing infection and/or re-infection wherein the patient is at risk due to altered stomach conditions.
